(19) United States
(12) Patent Application Publication
(10) Pub. No.: US 2024/0249554 A1
(43) Pub. Date: Jul. 25, 2024

(54) METHOD AND DEVICE FOR DETERMINING VISUAL FATIGUE OCCURRENCE SECTION

(71) Applicant: Emocog Inc., Seoul (KR)

(72) Inventor: Yoo Hun NOH, Seoul (KR)

(73) Assignee: Emocog Inc., Seoul (KR)

(21) Appl. No.: 18/627,581

(22) Filed: Apr. 5, 2024

Related U.S. Application Data
(63) Continuation of application No. PCT/KR2022/015073, filed on Oct. 7, 2022.

(30) Foreign Application Priority Data
Oct. 7, 2021 (KR) 10-2021-0133021

(51) Int. Cl. G06V 40/18; G06T 7/12; G06T 7/62

(52) U.S. Cl. CPC G06V 40/193; G06T 7/12; G06T 7/62; G06T 2207/30041

(57) ABSTRACT

A method of determining a visual fatigue occurrence duration includes: receiving an eye image that is an image including a plurality of frames and obtained by photographing an eye of a measurement target; determining a size of a pupil by analyzing the eye image; calculating each of a number of eye blinks, an eye closed time, and a pupil size change speed on the basis of an unspecified number of times and a change in the size of the pupil, in some frames of the determined pupil in the eye image; and determining a duration in which visual fatigue of the measurement target occurs, from among the plurality of frames, as a result of combining information regarding the calculated number of eye blinks, eye closed time, and pupil size change speed.

### CROSS-REFERENCE TO RELATED APPLICATIONS

This application is a continuation of International Application No. PCT/KR2022/015073 filed on Oct. 7, 2022, which claims priority to Korean Patent Application No. 10-2021-0133021 filed on Oct. 7, 2021, the entire contents of which are herein incorporated by reference.

### TECHNICAL FIELD

The present disclosure relates to a method of determining a visual fatigue occurrence duration and an apparatus therefor, and more particularly, to a method of determining a fatigue occurrence duration in which an actual fatigue feeling starts to accumulate in eyes of a user when the eyes of the user are exposed to an image that puts a strain on the eyes of the user and an apparatus for implementing the method.

### BACKGROUND ART

Modern people spend most of remaining time, excluding sleeping time, on using various types of smart devices. Staring at output units (screens) of electronic devices such as smart devices for a long time causes high visual fatigue to users.

In modern society, using smart devices is essential to collect information, and the time spent on using smart devices may not be blindly reduced, and thus, research has been actively conducted to measure the visual fatigue of users accumulated while using smart devices and reduce the same.

Various methods of effectively measuring visual fatigue occurring when users stare at displays of smart devices for a long period of time are known.

For example, there are methods of measuring visual fatigue by using the results of questionnaires for users, but these methods not only involve subjective evaluations by the users, but also have limitations that are difficult to quantify the visual fatigue to a degree that may be generalized according to the physical health of users.

As another example, there are methods of determining visual fatigue of users by measuring biometric signals of the users, such as electroencephalography (EEG), electro-cardiogram (ECG), and photo-plethysmogram (PPG), and analyzing the measured signals, but these methods have limitations in that the inconvenience of the users is great because unique equipment for measuring biometric signals is essential and various sensors need to be attached to the bodies of the users.

As another example, there are methods of measuring visual fatigue by using eye-trackers, but these methods demand full cooperation from users and thus has limitations in that data may not be collected simply.

As recently known methods, there are methods of measuring visual fatigue by measuring the number of eye blinks simultaneously while conducting questionnaires and combining the two, and the methods also include all of the limitations described above.

### SUMMARY

### Technical Problem

The technical problem to be solved by the present disclosure is to provide a method of accurately determining a duration in which visual fatigue occurs to a user by analyzing an eye image obtained by photographing an eye of the user and an apparatus for implementing the method.

### Technical Solution

A method according to an embodiment of the present disclosure for solving the above technical problem includes: receiving an eye image that is an image including a plurality of frames and obtained by photographing an eye of a measurement target; determining a size of a pupil by analyzing the eye image; calculating each of a number of eye blinks, an eye closed time, and a pupil size change speed on the basis of an unspecified number of times and a change in the size of the pupil, in some frames of the determined pupil in the eye image; and determining a duration in which visual fatigue of the measurement target occurs, from among the plurality of frames, as a result of combining information regarding the calculated number of eye blinks, eye closed time, and pupil size change speed.

An apparatus according to another embodiment of the present disclosure for solving the above technical problem includes: an image receiver configured to receive an eye image that is an image including a plurality of frames and obtained by photographing an eye of a measurement target; a pupil size determiner configured to determine a size of a pupil by analyzing the eye image; a parameter calculator configured to calculate each of a number of eye blinks, an eye closed time, and a pupil size change speed on the basis of an unspecified number of times and a change in the size of the pupil, in some frames of the determined pupil in the eye image; and a fatigue occurrence duration determiner configured to determine a duration in which visual fatigue of the measurement target occurs, from among the plurality of frames, as a result of combining information regarding the calculated number of eye blinks, eye close time, and pupil size change speed.

An embodiment of the present disclosure may provide a computer-readable recording medium storing a program for executing the method.

### Effects of Present Disclosure

According to the present disclosure, a point in time when fatigue accumulates in eyes of a user may be accurately determined.

In addition, according to the present disclosure, much more objective and accurate results may be calculated than measuring visual fatigue by attaching excessive sensors to the body of the user or performing a questionnaire.

### BRIEF DESCRIPTION OF DRAWINGS

FIG. 1 is a block diagram illustrating an example of an apparatus for determining a visual fatigue occurrence duration, according to the present disclosure.

FIG. 2A, FIG. 2B, FIG. 2C and FIG. 2D are views illustrating a process in which a pupil size determiner determines a pupil size of a measurement target.

FIG. 3 schematically illustrates an example of data referred to by a parameter calculator to calculate a pupil size change speed.

FIG. 4 illustrates a frequency domain graph of a pupil size change function when a threshold value is 0.5.

FIG. 5 illustrates a frequency domain graph of a pupil size change function when a threshold value is 0.1.

FIG. 6 illustrates a frequency domain graph of a pupil size change function when a threshold value is 0.01.

FIG. 7 is a graph illustrating a change in the number of eye blinks calculated by a parameter calculator.

FIG. 8 is a graph illustrating a change in an eye closed time calculated by a parameter calculator.

FIGS. 9 to 11 illustrate results of inversely transforming the frequency domain graphs described with reference to FIGS. 4 to 6 into time domain graphs.

FIG. 12 is a view schematically illustrating an example of a graph of a pupil size change speed calculated by a parameter calculator and analyzed by a fatigue occurrence duration determiner.

FIG. 13 is a flowchart illustrating an example of a method of determining a visual fatigue occurrence duration, according to the present disclosure.

### DETAILED DESCRIPTION

A method according to an embodiment of the present disclosure for solving the above technical problem includes: receiving an eye image that is an image including a plurality of frames and obtained by photographing an eye of a measurement target; determining a size of a pupil by analyzing the eye image; calculating each of a number of eye blinks, an eye closed time, and a pupil size change speed on the basis of an unspecified number of times and a change in the size of the pupil, in some frames of the determined pupil in the eye image; and determining a duration in which visual fatigue of the measurement target occurs, from among the plurality of frames, as a result of combining information regarding the calculated number of eye blinks, eye closed time, and pupil size change speed.

The determining of the duration in which the visual fatigue occurs may include combining a tendency to gradually increase the calculated number of eye blinks and a tendency to gradually increase the eye closed time, comparing a result of the combination with a preset condition, and determining, from among the plurality of frames, the duration in which the visual fatigue occurs.

The determining of the duration in which the visual fatigue occurs may include determining a reduction duration in which the calculated pupil size change speed is decreased and determining, from among the plurality of frames, the duration in which the visual fatigue of the measurement target occurs, on the basis of the determined reduction section.

The determining of the duration in which the visual fatigue occurs may include determining the duration in which the visual fatigue of the measurement target occurs, from among the plurality of frames, as a result of combining the tendency to gradually increase the calculated number of eye blinks, the tendency to gradually increase the eye closed time, and the reduction duration in which the calculated pupil size change speed is decreased.

The determining of the size of the pupil may include: extracting, from among the plurality of frames, a frame in which the eye of the measurement target is not closed; detecting at least one candidate group by applying binarization and contour detection techniques to the extracted frame; removing, from among the detected candidate group, reflected light caused by infrared light; and measuring a diameter of the pupil by considering a distribution of black pixels from among the candidate group from which the reflected light is removed.

An apparatus according to another embodiment of the present disclosure for solving the above technical problem includes: an image receiver configured to receive an eye image that is an image including a plurality of frames and obtained by photographing an eye of a measurement target; a pupil size determiner configured to determine a size of a pupil by analyzing the eye image; a parameter calculator configured to calculate each of a number of eye blinks, an eye closed time, and a pupil size change speed on the basis of an unspecified number of times and a change in the size of the pupil, in some frames of the determined pupil in the eye image; and a fatigue occurrence duration determiner configured to determine a duration in which visual fatigue of the measurement target occurs, from among the plurality of frames, as a result of combining information regarding the calculated number of eye blinks, eye close time, and pupil size change speed.

The fatigue occurrence duration determiner may be further configured to combine a tendency to gradually increase the calculated number of eye blinks and a tendency to gradually increase the eye closed time, compare a result of the combination with a preset condition, and determine, from among the plurality of frames, the duration in which the visual fatigue occurs.

The fatigue occurrence duration determiner may be further configured to determine a reduction duration in which the calculated pupil size change speed is decreased and determine, from among the plurality of frames, the duration in which the visual fatigue of the measurement target occurs, on the basis of the determined reduction section.

The fatigue occurrence duration determiner may be further configured to determine the duration in which the visual fatigue of the measurement target occurs, from among the plurality of frames, as a result of combining the tendency to gradually increase the calculated number of eye blinks, the tendency to gradually increase the eye closed time, and the reduction duration in which the calculated pupil size change speed is decreased.

The pupil size determiner may be further configured to extract, from among the plurality of frames, a frame in which the eye of the measurement target is not closed, detect at least one candidate group by applying binarization and contour detection techniques to the extracted frame, remove, from among the detected candidate group, reflected light caused by infrared light, and measure a diameter of the pupil by considering a distribution of black pixels from among the candidate group from which the reflected light is removed.

An embodiment of the present disclosure may provide a computer-readable recording medium storing a program for executing the method.

### Mode for Implementing Present Disclosure

The present disclosure may be modified in various ways and may have various embodiments, particular embodiments will be illustrated in the drawings and described in detail in the description. Advantages and features of the present disclosure, and methods of achieving the same will become clear with reference to the description of embodiments taken in conjunction with the accompanying drawings. However, it should be understood that the present disclosure is not limited to embodiments presented below, but may be implemented in various different forms.

Hereinafter, embodiments of the present disclosure will be described in detail with reference to the accompanying drawings, and when describing with reference to the drawings, identical or corresponding components will be assigned the same reference numerals and the same description thereof will be omitted.

In the following embodiments, the terms first, second, etc. may be only used herein to distinguish one component from another, not for limited sense.

In the following embodiments, the singular forms are intended to include the plural forms as well, unless the context clearly indicates otherwise.

The terms “comprise”, “include”, and/or “have” when used herein, specify the presence of stated features, elements, and/or components, but do not preclude the presence or addition of one or more other features, elements, and/or components.

In the cases where an embodiment may be implemented differently, a particular process sequence may be performed differently from the described sequence. For example, two processes described in succession may be performed substantially at the same time, or may be performed in an order opposite to the order described.

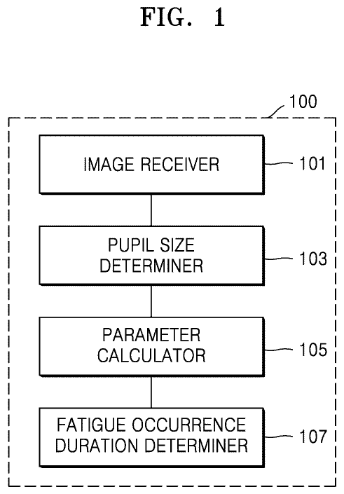

FIG. 1 is a block diagram illustrating an example of an apparatus for determining a visual fatigue occurrence duration, according to the present disclosure.

As shown in FIG. 1, a visual fatigue occurrence duration determination apparatus 100 according to the present disclosure may include an image receiver 101, a pupil size determiner 103, a parameter calculator 105, and a fatigue occurrence duration determiner 107. Hereinafter, for convenience of description, the visual fatigue occurrence duration determination apparatus 100 is referred to as a fatigue determination apparatus 100.

Names of modules included in the fatigue determination apparatus 100 shown in FIG. 1 are randomly named to intuitively describe a representative function performed by each module, and when the fatigue determination apparatus 100 according to the present disclosure is physically or logically implemented, each module may be given a different name from the name written in FIG. 1.

In addition, the number of modules included in the fatigue determination apparatus 100 of FIG. 1 may vary each time according to an embodiment. In more detail, the fatigue determination apparatus 100 of FIG. 1 includes a total of four modules, from the image receiver 101 to the fatigue occurrence duration determiner 107, but according to an embodiment, at least one module may be integrated into another module, or at least one module may be implemented as separated into two or more modules.

In addition, the image receiver 101, the pupil size determiner 103, the parameter calculator 105, and the fatigue occurrence duration determiner 107 included in the fatigue determination apparatus 100 of FIG. 1 may correspond to at least one processor or may include at least one processor. Accordingly, the fatigue determination apparatus 100 may be driven in a form included in another hardware apparatus such as a microprocessor or a general-purpose computer system.

The fatigue determination apparatus 100 shown in FIG. 1 highlights and illustrates only components for representing characteristics of an embodiment of the present disclosure. Therefore, according to an embodiment different from the embodiment shown in FIG. 1, one of ordinary skill in the art may understand that other general-purpose components may be further included in addition to the components shown in FIG. 1.

The image receiver 101 receives an eye image. The eye image is an image including a plurality of frames, and refers to an image obtained by photographing an eye of a measurement target. The eye image may be an image of an eye rather than eyes of the measurement target.

The eye image received by the image receiver 101 includes a plurality of frames and thus is data in the form of a video rather than a still image, and may be, for example, a video including of 50000 frames to 100000 frames. A frame rate of the eye image may be 30 frames or more per second to accurately determine visual fatigue accumulated in the measurement target.

As another example, the eye image may be an image captured by an infrared camera of an HQCAM passing infrared rays having a wavelength of 750 nm or more. In addition, the wavelength of the infrared light used may be 850 nm, and the wavelength of the infrared light as described above guarantees high clarity of the eye image. In addition, the image receiver 101 may receive the eye image from an external apparatus by wired or wireless.

The pupil size determiner 103 determines a size of a pupil by analyzing the eye image. Here, the analysis of the eye image comprehensively expresses a process of pre-processing the eye image to determine the size of the pupil in the eye image.

The pupil size determiner 103 may include a memory that may temporarily store some frames of the eye image to analyze the eye image and a processor that may process an algorithm for analyzing the eye image. Since in the eye image, other skin around the eye is also together captured in addition to the eye of the measurement target, only the pupil needs to be determined in the eye image to accurately analyze the eye image, and the pupil size determiner 103 may determine a location and size of the pupil in the eye image through various types of algorithms. When the measurement target closes the eyes thereof, the location and size of the pupil may not be determined, and the pupil size determiner 103 stores numbers of frames and a number of frames, in which the location and size of the pupil are not determined and use the same for calculating the number of eye blinks and an eye closed time described below.

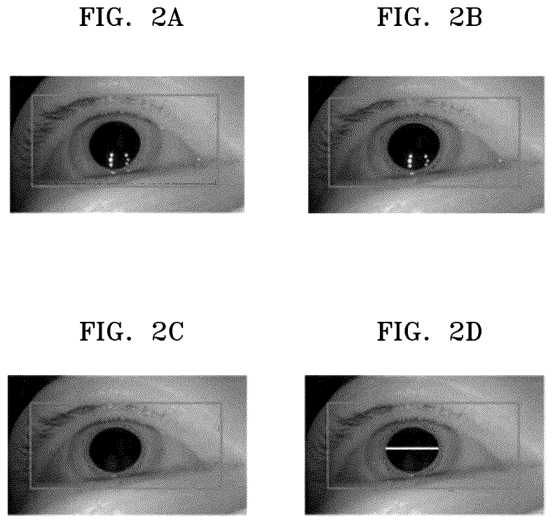

FIG. 2A, FIG. 2B, FIG. 2C and FIG. 2D are views illustrating a process in which the pupil size determiner 103 determines a pupil size of a measurement target.

FIG. 2 illustrates four frames for describing a process of determining a location and a size of a pupil. First, FIG. 2A shows that an eye area is determined to detect the pupil. In FIG. 2A, a known facial recognition algorithm may be used to measure the eye area of the measurement object. The known facial recognition algorithm may accurately determine a location of an eyeball in an eye image by considering a shape and a sense of volume, and in the case where the facial recognition algorithm does not operate, the eye area may be directly designated through an input from a user.

FIG. 2B illustrates a process of finding a pupil area by using binarization and contour detection techniques in a primarily designated eye area. The pupil size determiner 103 may select a plurality of candidate group frames to find the pupil area. In this process, the pupil size determiner 103 may use an aspect ratio and a minimum pupil size.

FIG. 2C illustrates a frame in which reflected light generated by infrared light is removed from the detected pupil area. Referring to FIG. 2C, in the pupil from which the reflected light is removed, black pixels are concentrated in a circular shape along a shape of the pupil.

FIG. 2D is a view illustrating a process of determining a size of a pupil by considering, as a diameter of the pupil, a row having the greatest number of black pixels in the pupil area from which the reflected light is removed. The size of the pupil is calculated in units of frames over time, and for example, 54000 pupil sizes may be obtained from a 30-minute-long eye image captured at 30 frames per second (fps).

Subsequently, the parameter calculator 105 calculates each of the number of eye blinks, an eye closed time, and a pupil size change speed on the basis of the unspecified number of times in some frames of the pupil determined in the eye image and a change in size of the pupil. Parameters calculated by the parameter calculator 105 are a total of three types, are the number of eye blinks, the eye closed time, and the pupil size change speed, and are parameters calculated for all frames (e.g., 54000) that constitute the eye image, not for a single frame.

In detail, the parameters calculated by the parameter calculator 105 may be information regarding the number of eye blinks, information regarding the eye closed time, and information regarding an increase or decrease state in the pupil size change speed.

As an example, the parameter calculator 105 may calculate information regarding a tendency to gradually increase the number of eye blinks and a tendency to gradually increase the eye closed time.

As another example, the parameter calculator 105 may determine, in a plurality of frames, a reduction duration in which the pupil size change speed decreases, calculate the determined information as one parameter, and transmit the calculated one parameter to the fatigue occurrence duration determiner 107 described below.

To measure the information regarding the number of eye blinks of the measurement target, the parameter calculator 105 may measure the number of eye blinks by counting frequencies at which the pupil size is 0 in frames in which the pupil size is not 0. Here, the pupil size being 0 indicates that the measurement target temporarily closes eyes and thus the pupil is not confirmed by an infrared camera that captures an eye image, and the number of times the pupil size is 0 may be referred to as an unspecified number of times of the pupil size.

The parameter calculator 105 may calculate the information regarding the eye-closed time by counting the unspecified number of times of the pupil size and considering a frame rate of the eye image.

The parameter calculator 105 may calculate the pupil size change speed on the basis of the location and size of the pupil determined by the pupil size determiner 103.

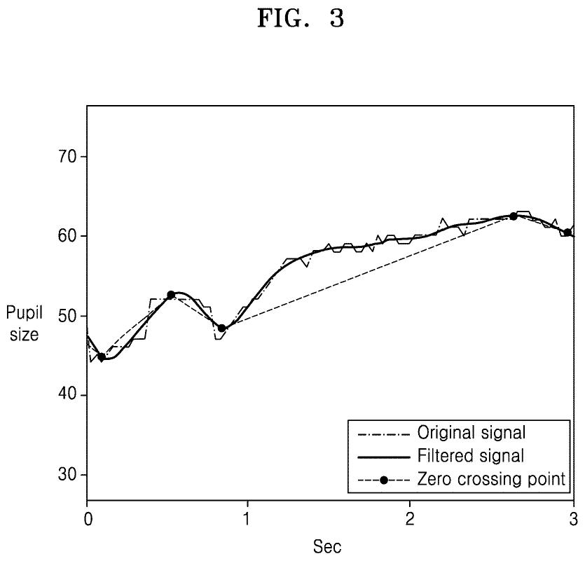

FIG. 3 schematically illustrates an example of data referred to by the parameter calculator 105 to calculate a pupil size change speed.

A blue graph of FIG. 3 shows a change in pupil size in each frame. Referring to FIG. 3, the blue graph shows a change in pupil size for three seconds, and in the case of an eye image of 30 fps, shows the change in pupil size in a total of 90 frames. When the present disclosure is actually implemented, in the case where the eye image includes 54000 frames, a time axis of the graph of FIG. 3 may further extend to 1800 seconds.

A yellow graph of FIG. 3 shows a graph in which high-frequency noise is removed from the blue graph. The blue graph has noise such as slight tremors or bouncing values, and a result of filtering a function of the blue graph through fast Fourier transform (FFT) is the yellow graph. The parameter calculator 105 may set a threshold value to effectively remove noise including a high-frequency component from the blue graph. Here, the threshold value is a variable that may be changed mathematically, experimentally, or empirically to maximize a filtering effect. The parameter calculator 105 may calculate a function, which is a base of the yellow graph, by applying FFT to the function of the blue graph corresponding to an original signal, removing a high-frequency component above the threshold value, and applying inverse fast Fourier transform (IFFT) again.

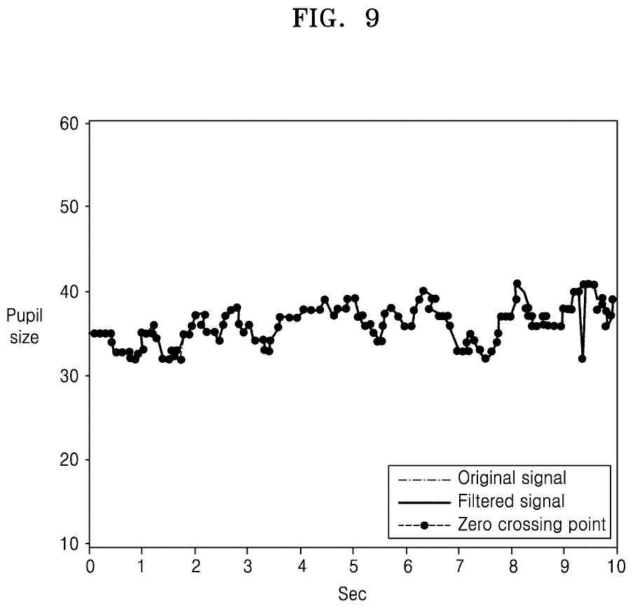

In FIG. 3, a zero crossing point refers to an inflection point at which development of a change in pupil size is changed on the yellow graph from which the high-frequency noise is removed. The zero crossing point of FIG. 3 is described in detail with reference to FIGS. 9 to 11.

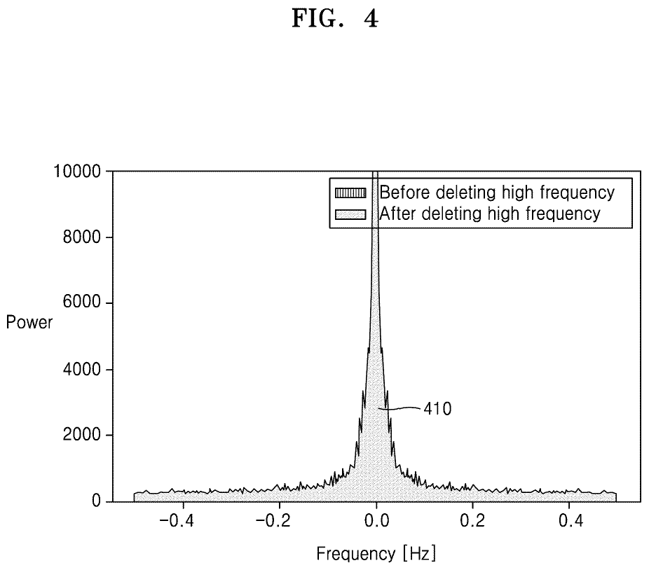

FIG. 4 illustrates a frequency domain graph of a pupil size change function when a threshold value is 0.5.

In more detail, FIG. 4 shows a graph with respect to magnitude and frequency by applying FFT to the functions of the blue graph and the yellow graph of FIG. 3 corresponding to original data. A graph of FIG. 4 formed by a blue line corresponds to the blue graph of FIG. 3, and a graph of FIG. 4 formed by a yellow line corresponds to the yellow graph of FIG. 3. Comparing a filter result 410 of FIG. 4 with the blue graph, a lot of noise is removed in a wide frequency domain.

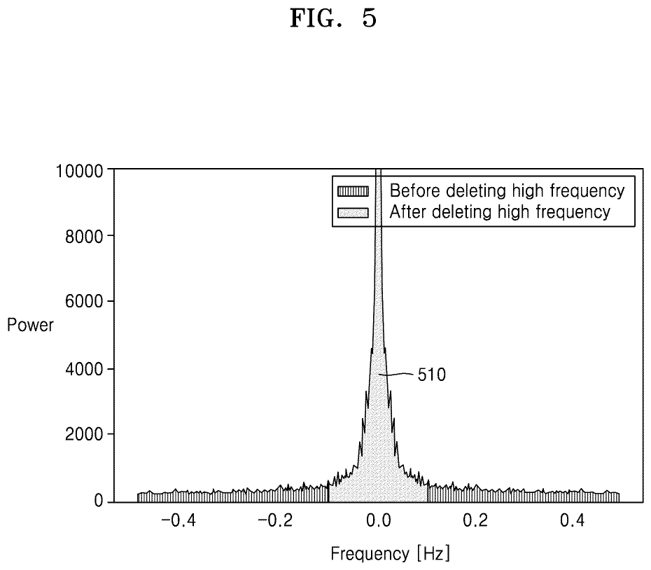

FIG. 5 illustrates a frequency domain graph of a pupil size change function when a threshold value is 0.1.

Comparing FIG. 5 with FIG. 4, when the threshold value is reduced from 0.5 to 0.1, an x axis of a yellow graph is limited from −0.1 Hz to 0.1 Hz. A filter result 510 when the threshold value is 0.1 schematically shows that not only high frequency noise but also low frequency noise is removed.

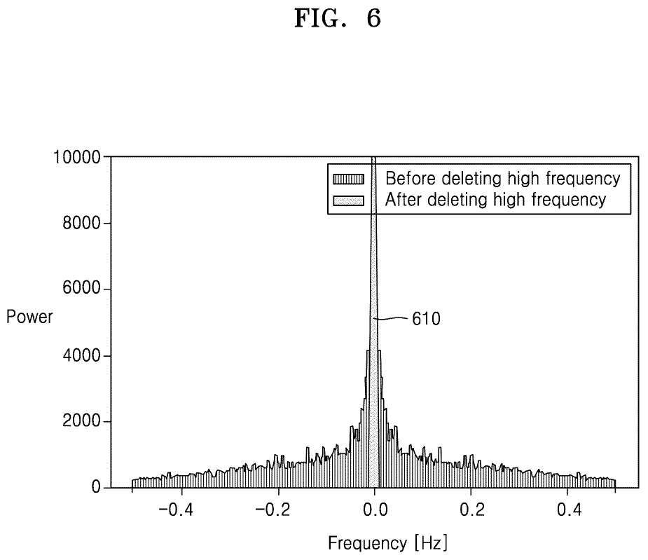

FIG. 6 illustrates a frequency domain graph of a pupil size change function when a threshold value is 0.01.

Comparing FIG. 6 with FIGS. 4 and 5, when the threshold value is further reduced from 0.5 to 0.01, an x axis of a yellow graph is limited from −0.01 Hz to 0.01 Hz. In a filter result 610 when the threshold value is 0.01, noise is removed across all frequency domains.

Through the above method, the parameter calculator 105 calculates each of the number of eye blinks, an eye closed time, and a pupil size change speed with respect to a measurement target, and for analysis of the same, transmits the calculated parameters to the fatigue occurrence duration determiner 107.

As a result of combining information regarding the number of eye blinks, the eye closed time, and the pupil size change speed calculated by the parameter calculator 105, the fatigue occurrence duration determiner 107 determines a duration in which visual fatigue of the measurement target occurs, from among a plurality of frames constituting an eye image.

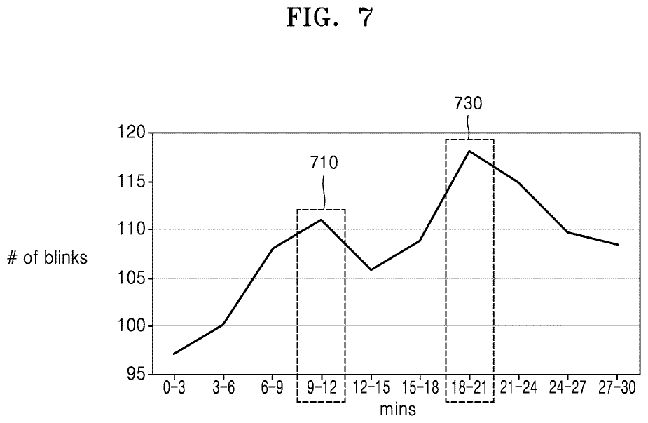

FIG. 7 is a graph showing a change in the number of eye blinks calculated by a parameter calculator.

In detail, FIG. 7 shows the average number of eye blinks extracted from 10 measurement targets for 30 minutes. A horizontal axis of FIG. 7 forms one duration every three minutes, several durations are consecutively connected to one another, and a unit is minutes. In FIG. 7, a first duration to a tenth duration are present, and inflection points of the number of eye blinks occur in a fourth duration 710 and a seventh duration 730.

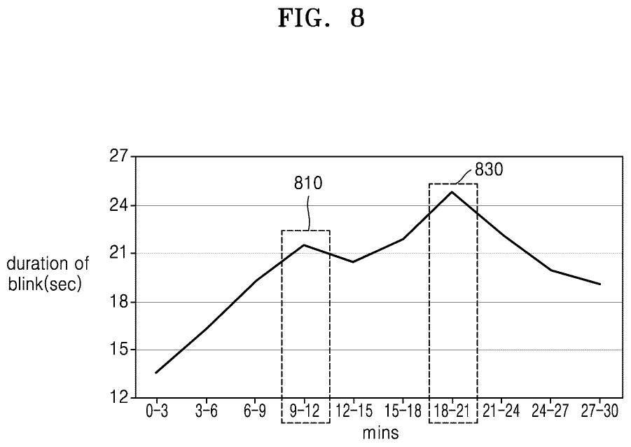

FIG. 8 is a graph showing a change in an eye closed time calculated by a parameter calculator.

In detail, referring to FIG. 8, in a fourth duration 810, the eye closed time increases to 21 seconds, and in a seventh duration 830, the eye closed time increases again to 25 seconds and then starts to decrease.

The fatigue occurrence duration determiner 107 comprehensively interprets FIGS. 7 and 8 and determines, on the basis that inflection points occur both in a fourth duration and a seventh duration, visual fatigue occurs above a certain level in the corresponding durations. Here, in the present disclosure, visual fatigue does not simply indicate whether or not a strain is put on eyes of a measurement target, but is regarded as fatigue at a level at which the measurement target (a user) actually feels fatigue when the strain felt on the eyes exceeds a predetermined reference fatigue value.

The results as in FIGS. 7 and 8 indicate that in general, visual fatigue felt by the user does not only increase linearly, but also a duration is present in which the fatigue felt by the user is relieved through saturation and adaptation after a certain period of time elapses or a certain amount of fatigue is accumulated.

As described above, the fatigue occurrence duration determiner 107 determines a duration in which visual fatigue occurs, by using a pupil size change speed as one parameter in addition to the number of eye blinks and an eye closed time.

FIGS. 9 to 11 illustrate results of inversely transforming the frequency domain graphs described with reference to FIGS. 4 to 6 into time domain graphs.

The parameter calculator 105 obtains an inflection point from a noise-filtered graph to calculate a pupil size change speed (a pupil size change rate). A zero crossing point may be used as a method for the parameter calculator 105 to obtain the inflection point. The pupil size change speed may be obtained by dividing a pupil size difference between two inflection points by a difference in the number of frames of the two inflection points, and the parameter calculator 105 may calculate the pupil size change speed by a method of averaging speeds between two zero crossing points calculated as described above in units of durations (e.g., 3minutes).

First, in the case of FIG. 9, a threshold value for removing noise is set to 0.5, and thus, noise is not almost removed and a plurality of zero crossing points are formed.

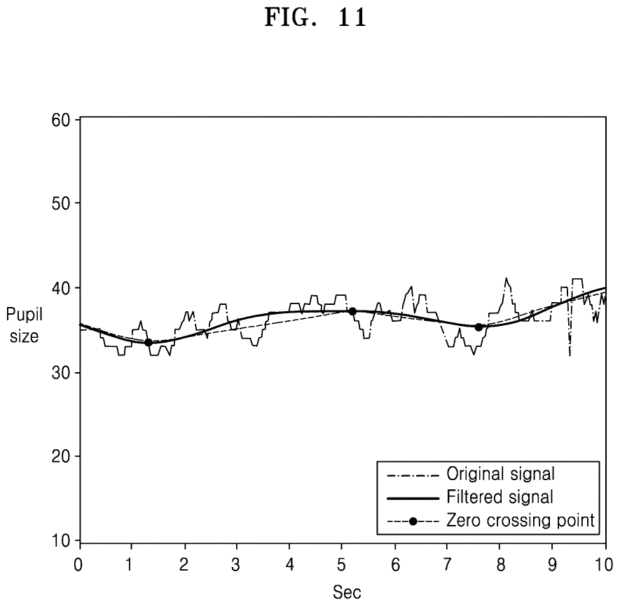

In addition, in the case of FIG. 11, a threshold value for removing noise is set to 0.01, and thus, only a small number of zero crossing points, which are insufficient to be analyzed, are formed because noise is excessively removed and few actual components are left.

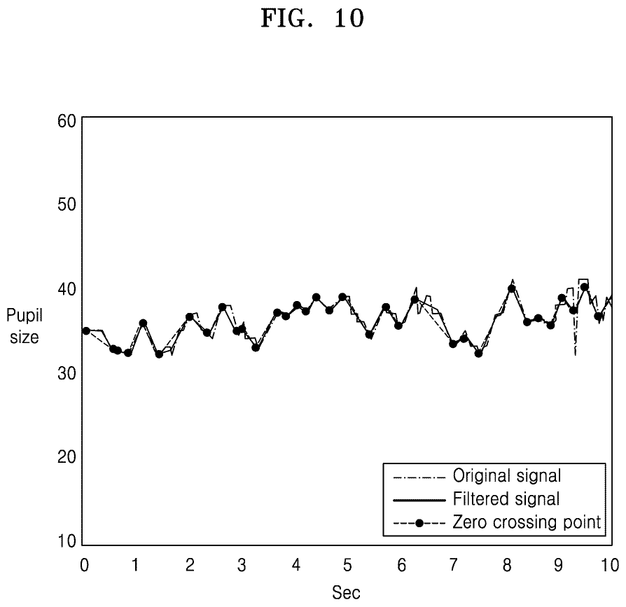

As a result, as on the graph of FIG. 10, the parameter calculator 105 may calculate the above-described pupil size change speed from a graph in which zero crossing points are formed to a level appropriate for analysis. When the present disclosure is actually implemented, the method may be implemented in a method by which the parameter calculator 105 calculates zero crossing points by dividing into several cases for respectively a plurality of preset threshold values and calculates pupil size change speeds for the respective calculated zero crossing points or outputs the respective calculated pupil size change speeds to allow the user to select one of the pupil size change speeds.

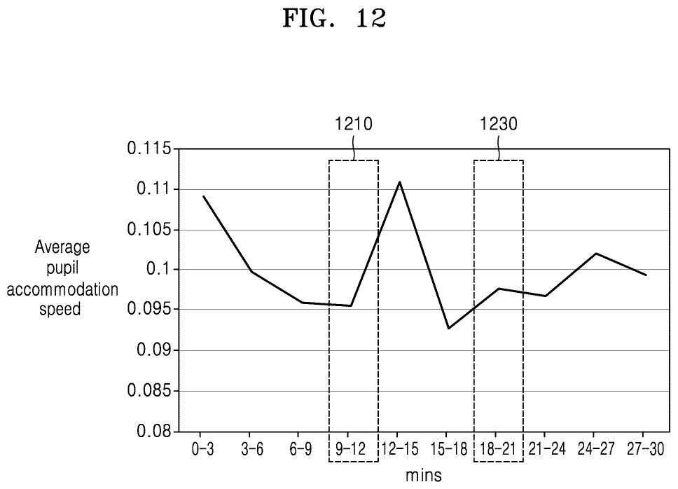

FIG. 12 is a view schematically illustrating an example of a graph of a pupil size change speed calculated by a parameter calculator and analyzed by a fatigue occurrence duration determiner.

As in FIGS. 7 and 8, FIG. 12 also illustrates that 10 3-minute unit durations are present, and referring to FIG. 7, a pupil size change speed decreases in a fourth duration 1210 and a seventh duration 1230. A horizontal axis of FIG. 12 represents time and a vertical axis represents a pupil size change speed. In particular, the horizontal axis is divided into units of durations as in FIGS. 7 and 8.

The fatigue occurrence duration determiner 107 may comprehensively analyze FIGS. 7, 8, and 12 to determine that visual fatigue of a user occurs in 9 minutes to 12 minutes and 18 minutes to 21 minutes corresponding to a fourth duration and a seventh duration. As described above, the present disclosure may calculate inflection points at which the number of eye blinks and an eye closed time increase and then suddenly decrease and an inflection point at which a pupil size change speed gradually decreases and then suddenly increases, and in the case where the respective calculated inflection points match one another when combined, determine that visual fatigue of a measurement target reaches a certain level or more.

As a selective embodiment, the fatigue occurrence duration determiner 107 may determine a visual fatigue occurrence duration of a measurement target only in the case where durations of inflection points of the number of eye blinks, an eye closed time, and a pupil size change speed match one another. In other words, the fatigue occurrence duration determiner 107 may determine that the visual fatigue does not occur when the inflection point of any one of the number of eye blinks, the eye closed time, and the pupil size change speed does not match the remaining inflection points.

As described above, the fatigue occurrence duration determiner 107 may adjust the number of blinks, the eye closed time, and the pupil size change speed calculated by the parameter calculator 105 on a time (duration) basis to additionally configure tendencies or inflection points, determine a visual fatigue occurrence duration on the basis of the configured values.

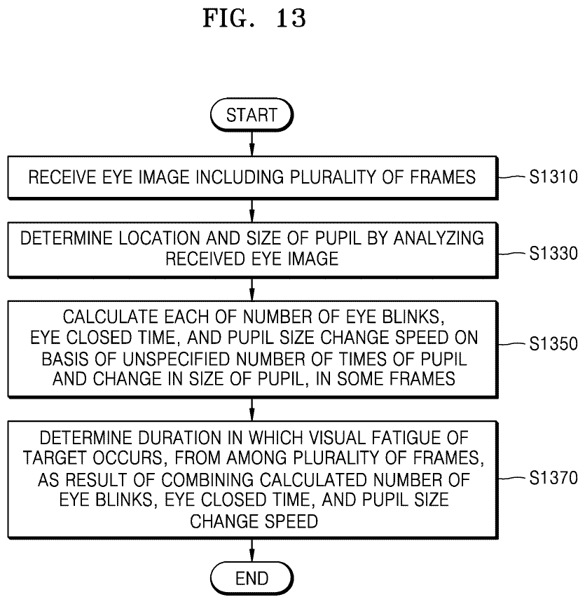

FIG. 13 is a flowchart illustrating an example of a method of determining a visual fatigue occurrence duration, according to the present disclosure.

The method of FIG. 13 may be implemented by the fatigue determination apparatus 100 according to FIG. 1, and thus, the same descriptions of FIG. 13 as the descriptions of FIGS. 1 to 12 are omitted below.

In operation S1310, the image receiver 101 receives an eye image including a plurality of frames.

In operation S1330, the pupil size determiner 103 analyzes the received eye image and determines a size of a pupil.

In operation S1350, the parameter calculator 105 calculates each of the number of eye blinks, an eye closed time, and a pupil size change speed, on the basis of the unspecified number of times of a pupil and changes in size of the pupil, in some frames.

In operation S1370, the fatigue occurrence duration determiner 107 may determine a duration in which visual fatigue of a measurement target occurs, from among a plurality of frames, as a result of combining three parameters (the number of eye blinks, the eye closed time, and the pupil size change speed) calculated in operation S1350. When operation S1370 is applied to FIGS. 7, 8, and 12, the visual fatigue of the measurement target may be interpreted as occurring above a preset reference fatigue value in the fourth duration (9 minutes to 12 minutes) and the seventh duration (18 minutes to 21 minutes).

In FIGS. 7, 8, and 12, for convenience of description, a 30-minute eye image is used, but when a length of the eye image further increases, a third inflection duration, a fourth inflection duration, and the like may be further observed in addition to a first inflection duration (the fourth duration) and a second inflection duration (the seven duration), and the fatigue occurrence duration determiner 107 may determine the occurrence of visual fatigue on the basis of additionally observed inflection durations.

According to the present invention, a duration in which visual fatigue of a user as a measurement target occurs may be accurately determined only with an eye image captured for a relatively short time.

In the present disclosure, to accurately determine the duration in which the visual fatigue of the user occurs, a size of a pupil may be determined, the number of eye blinks, an eye closed time, and a pupil size change speed may be calculated, and then a result of analyzing a combination of the three parameters may be used.

Embodiments according to the present disclosure described above may be implemented in the form of a computer program that may be executed through various components on a computer, and the computer program may be recorded on a computer-readable medium. Here, the medium may include magnetic media, such as a hard disk, a floppy disk, and a magnetic tape, optical recording media, such as CD-ROM and DVD, magneto-optical media, such as a floptical disk, and hardware devices, such as ROM, RAM, and flash memory devices specially configured to store and execute program instructions.

Meanwhile, the computer program may be specially designed and configured for the present disclosure, or may be known to and used by those skilled in the art of the computer software field. Examples of the computer program may include not only machine language code generated by a compiler but also high-level language code that may be executed by a computer by using an interpreter or the like.

The particular implementations described in the present disclosure are embodiments and do not limit the scope of the present disclosure in any way. For brevity of the description, descriptions of existing electronic components, control systems, software, and other functional aspects of the systems may be omitted. In addition, connections or connection members of lines between components shown in the drawings are only examples of functional connections and/or physical or circuit connections, and in an actual apparatus, connections between components may be represented by various functional connections, physical connections, or circuit connections that may be replaced or added. In addition, when there is no particular mention, such as “essentially” or “importantly”, it may not be an essential component for the application of the present disclosure.

In the description of the present disclosure (particularly, in claims), the use of the term “the” and similar indicative terms may correspond to both the singular and the plural forms. In addition, when a range is described in the present disclosure, the present disclosure includes the application of individual values within the range (unless there is a statement to the contrary), and each individual value constituting the range is described in the detailed description of the present disclosure. The operations constituting the method according to the present disclosure may be performed in any appropriate order unless an order of the operations is explicitly stated or stated to the contrary. The present disclosure is not necessarily limited according to the order of description of the operations. The use of all examples or example terms (e.g., and the like) in the present disclosure is simply to describe the present disclosure in detail, and the scope of the present disclosure is limited due to the examples or example terms unless limited by claims. In addition, those skilled in the art may appreciate that various modifications, combinations and changes may be made according to design conditions and factors within the scope of the appended claims or equivalents thereof.

1. A method of determining a visual fatigue occurrence duration, the method comprising:
receiving an eye image that is an image comprising a plurality of frames and obtained by photographing an eye of a measurement target;
determining a size of a pupil by analyzing the eye image;
calculating each of a number of eye blinks, an eye closed time, and a pupil size change speed on the basis of an unspecified number of times and a change in the size of the pupil, in some frames where the size of the pupil in the eye image is determined; and
determining a duration in which visual fatigue of the measurement target occurs, from among the plurality of frames, as a result of combining information regarding the calculated number of eye blinks, eye closed time, and pupil size change speed.

2. The method of claim 1, wherein the determining of the duration in which the visual fatigue occurs comprises combining a tendency to gradually increase the calculated number of eye blinks and a tendency to gradually increase the eye closed time, comparing a result of the combination with a preset condition, and determining, from among the plurality of frames, the duration in which the visual fatigue occurs.

3. The method of claim 1, wherein the determining of the duration in which the visual fatigue occurs comprises determining a reduction duration in which the calculated pupil size change speed is decreased and determining, from among the plurality of frames, the duration in which the visual fatigue of the measurement target occurs, on the basis of the determined reduction duration.

4. The method of claim 1, wherein the determining of the duration in which the visual fatigue occurs comprises determining the duration in which the visual fatigue of the measurement target occurs, from among the plurality of frames, as a result of combining a tendency to gradually increase the calculated number of eye blinks, a tendency to gradually increase the eye closed time, and a reduction duration in which the calculated pupil size change speed is decreased.

5. The method of claim 1, wherein the determining of the size of the pupil comprises:
extracting, from among the plurality of frames, a frame in which the eye of the measurement target is not closed;
detecting at least one candidate group by applying binarization and contour detection techniques to an extracted frame;
removing, from among the at least one candidate group detected, reflected light caused by infrared light; and
measuring a diameter of the pupil by considering a distribution of black pixels from among the candidate group from which the reflected light is removed.

6. A non-transitory computer-readable recording medium storing a program for executing the method of claim 1.

7. An apparatus for determining a visual fatigue occurrence duration, the apparatus comprising:
an image receiver configured to receive an eye image that is an image comprising a plurality of frames and obtained by photographing an eye of a measurement target;
a pupil size determiner configured to determine a size of a pupil by analyzing the eye image;
a parameter calculator configured to calculate each of a number of eye blinks, an eye closed time, and a pupil size change speed on the basis of an unspecified number of times and a change in the size of the pupil, in some frames where the size of the pupil in the eye image is determined; and
a fatigue occurrence duration determiner configured to determine a duration in which visual fatigue of the measurement target occurs, from among the plurality of frames, as a result of combining information regarding the calculated number of eye blinks, eye close time, and pupil size change speed.

8. The apparatus of claim 7, wherein the fatigue occurrence duration determiner is further configured to combine a tendency to gradually increase the calculated number of eye blinks and a tendency to gradually increase the eye closed time, compare a result of the combination with a preset condition, and determine, from among the plurality of frames, the duration in which the visual fatigue occurs.

9. The apparatus of claim 7, wherein the fatigue occurrence duration determiner is further configured to determine a reduction duration in which the calculated pupil size change speed is decreased and determine, from among the plurality of frames, the duration in which the visual fatigue of the measurement target occurs, on the basis of the determined reduction duration.

10. The apparatus of claim 7, wherein the fatigue occurrence duration determiner is further configured to determine the duration in which the visual fatigue of the measurement target occurs, from among the plurality of frames, as a result of combining a tendency to gradually increase the calculated number of eye blinks, a tendency to gradually increase the eye closed time, and a reduction duration in which the calculated pupil size change speed is decreased.

11. The apparatus of claim 7, wherein the pupil size determiner is further configured to
extract, from among the plurality of frames, a frame in which the eye of the measurement target is not closed,
detect at least one candidate group by applying binarization and contour detection techniques to an extracted frame,
remove, from among the at least one candidate group detected, reflected light caused by infrared light, and
measure a diameter of the pupil by considering a distribution of black pixels from among the at least one candidate group from which the reflected light is removed.
